(19) United States
(12) Patent Application Publication
(10) Pub. No.: US 2025/0134385 A1
(43) Pub. Date: May 1, 2025

(54) METHODS AND INSTRUMENTS FOR MONITORING MENSTRUAL FLUID FOR DIAGNOSIS AND TREATMENT

(71) Applicants: Nikita Prabhakar, Madison, AL (US); Ashwin Prabhakar, Madison, AL (US)

(72) Inventors: Nikita Prabhakar, Madison, AL (US); Ashwin Prabhakar, Madison, AL (US)

(21) Appl. No.: 18/925,980

(22) Filed: Oct. 24, 2024

Related U.S. Application Data
(60) Provisional application No. 63/593,951, filed on Oct. 27, 2023.

(51) Int. Cl. A61B 5/00; A61B 5/145

(52) U.S. Cl. CPC A61B 5/0082; A61B 5/0062; A61B 5/145; A61B 5/4839; A61B 5/6804; A61B 5/7264; A61B 2560/0462; A61B 2562/043

(57) ABSTRACT

According to an aspect of at least one embodiment, a method of monitoring menstrual fluid for diagnosis and treatment may include providing a menstrual hygiene product including a sensor. Data may be obtained from including data collected by the sensor. A menstrual characteristic may be determined based on the data. The menstrual characteristic may be monitored for a menstrual condition. A safety action may be performed based on fulfillment of the menstrual condition.

### CROSS-REFERENCE TO RELATED APPLICATIONS

This patent application claims priority to U.S. Provisional Application No. 63/593,951 filed Oct. 27, 2023, which provisional is incorporated herein by specific reference in its entirety.

### FIELD

The present disclosure relates to methods and instruments for monitoring menstrual fluid for diagnosis and treatment.

### BACKGROUND

Menstruation is the monthly shedding of the lining of the uterus. Menstruation is also known by the terms menses, menstrual period, menstrual cycle or period. Menstrual fluid-which is partly blood and partly tissue from the inside of the uterus-flows from the uterus through the cervix and out of the body through the vagina. For most women menstruation occurs every 28 days but menstruation may occur from around every 24 days to around every 38 days. A period can last between 2 and 7 days, but typically lasts for about 5 days. The bleeding tends to be heaviest in the first 2 days. Most women lose about between 30 and 40 milliliters, or two to three tablespoons, of blood during menstruation.

To make sure that the menstrual fluid does not soil clothes or other items, different types of menstrual hygiene products are used to absorb or collect the fluid. Menstrual hygiene products may include sanitary pads, tampons, and menstrual cups. Sanitary pads are strips of padding that may have an adhesive on one side that may be attached to the underwear of the user to hold the sanitary pad in place. A portion of the pad may be made of an absorbent material that soaks up the fluid. Pads come in many sizes, so the user may choose a pad to suit how heavy or light the user's period is. One can also use reusable washable pads or biodegradable pads made out of material like cotton, coconut, or banana fibers.

Pantyliners are a smaller and thinner type of sanitary pad that may be used on days when the period is very light. Tampons are small tubes typically made out of cotton that may be inserted into the vagina to absorb the menstrual fluid. Tampons may include an applicator to allow for insertion, or tampons may be provided without an applicator and may be inserted manually. In both cases, a string may be placed at one end of the tampon, which may be pulled to remove the tampon.

Menstrual cups are an alternative to sanitary pads and tampons. The cup may be made from silicone and may be inserted into the vagina. Menstrual cups collect the blood rather than absorb it. Unlike tampons and most sanitary pads, which are typically thrown away after use, menstrual cups may be washed and reused.

Period underwear are underwear made from absorbent fabric. Period underwear may soak up blood in the same way as sanitary pads and may include a layer to prevent leaks. Period underwear may be designed to be washed and reused.

Heavy menstrual bleeding (Menorrhagia) affects more than 10 million (one out of five) American women each year. Women who have menorrhagia may bleed for more than 7 days and lose more than 80 milliliters of blood. Menorrhagia may lead to anemia which may cause fatigue, weakness, and other physical issues. Blood loss in menorrhagia is currently diagnosed based on a menstrual chart and a scoring system. The menstrual chart and scoring system may include a questionnaire designed to elicit the number of pads used, the qualitative saturation level of the pad, the qualitative size of blood clots, and the duration of the menstruation. Each lightly soaked pad is given a score of 1 point, each moderately soaked pad is given a score of 5 points, and each heavily soaked pad is given a score of 20 points. Each lightly soaked tampon is given a score of 1 point, each moderately soaked pad is given a score of 5 points, and each heavily soaked tampon is given a score of 10 points. Finally, 1 point is given to small blood clots (grape size) and 5 points are given to large blood clots (strawberry size). A score of ≥100 points during menstruation indicates probable menorrhagia.

However, the menstrual chart and scoring system may be highly inaccurate as soak level and clot-size estimation are subjective and so estimates may vary significantly person-to-person. Additionally, some users may have issues identifying blood clots at all. Furthermore, the menstruation chart requires daily manual tracking from the user, and, as a result, the effectiveness is dependent on the user being consistent in utilizing the chart. The chart also does not allow the user to monitor treatments applied during menstruation. Because the menstrual chart and scoring system is based on manual tracking, subjective determination, and does not provide a way to monitor treatments applied during menstruation, the chart may lead to misdiagnosis and may not be an efficient way to monitor treatments applied during menstruation.

Additionally, menstrual fluid includes blood, old parts of uterine tissue, cells from the mucus lining of the vagina, and bacteria making up the vaginal flora. In addition, menstrual fluid may contain other biological components like nucleic acids, proteins, and metabolites. Several studies have been conducted to show that the components in menstrual fluid can correlate with components from regular blood draws. However, blood draws may be invasive to the patient and are also inconvenient.

Accordingly, there is a need for a menstrual hygiene product that provides a more convenient and non-invasive method for monitoring and analyzing menstrual fluids for diagnoses and treatment. Specifically, there is a need for real-time accurate quantitation of menstrual characteristics based on characteristics such as the volume of fluid absorbed by the menstrual hygiene product and the clot sizes of the blood captured by the sanitary product. In addition, there is a need for monitoring treatments during menstruation.

The subject matter claimed in the present disclosure is not limited to embodiments that solve any disadvantages or that operate only in environments such as those described above. Rather, this background is only to illustrate one example technology area where some embodiments described in the present disclosure may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used to limit the scope of the claimed subject matter.

Exemplary embodiments of the present disclosure address the limitations of traditional methods of monitoring menstruation. Embodiments disclosed herein address these issues by providing sensors incorporated in menstrual hygiene products allowing menstruation to be monitored without users having to manually track the menstruation and without invasive methods. According to an aspect of at least one embodiment, an instrument may include a menstrual hygiene product and a sensor incorporated in the menstrual hygiene product. The sensor may be configured to collect data from menstrual fluid contacting the menstrual hygiene product.

In some embodiments, the sensor may be configured to collect at least one of weight data, time data, resistance data, conductivity data, viscosity data, image data, or biological data of the menstrual fluid.

In some embodiments, the instrument may also include a camera incorporated in the menstrual hygiene product or an external camera. The camera may be configured to capture images of the menstrual fluid contacting the menstrual hygiene product.

In some embodiments, the instrument may also include a processor incorporated in the menstrual hygiene product. In these and other embodiments, the processor may be communicatively coupled to the sensor and the processor may be configured to transmit the data collected by the sensor to a user device. Additionally or alternatively, the processor may be communicatively coupled to the camera, and the processor may be configured to transmit images captured by the camera to the user device.

In some embodiments, the processor may be configured to determine a menstrual characteristic based on the data, wherein the menstrual characteristic includes at least one of blood loss, saturation level of the menstrual hygiene product, time that the menstrual hygiene product has been in use, a presence of biomarkers in the menstrual fluid, or blood clot size.

In these and other embodiments, the processor may be configured to monitor a menstrual characteristic for a menstrual condition, wherein the menstrual condition includes at least one of a blood loss threshold, a saturation threshold, a time threshold, a presence of a specific biomarker in the menstrual fluid, a blood clot size threshold, or a medication release time.

In some embodiments, the menstrual hygiene product may be configured to release medicine in response to receiving a signal from the user device to dispense the medicine, in response to the processor receiving data from the sensor indicating that a menstrual condition has been fulfilled, or in response to the sensor coming into contact with the menstrual fluid. In some embodiments, the menstrual hygiene product may be a tampon, a sanitary pad, period underwear, or a menstrual cup, as well as any other wearable article of manufacture. In some embodiments, the sensor may be a part of an array of sensors. The array of sensors may be incorporated into the menstrual hygiene product.

According to an aspect of at least one embodiment, a method of monitoring menstrual fluid for diagnosis and treatment may include providing a menstrual hygiene product including a sensor. Data may be obtained from the menstrual hygiene product including data collected by the sensor. A menstrual characteristic may be determined based on the data obtained. The menstrual characteristic may be monitored for a menstrual condition. A safety action may be performed based on fulfillment of the menstrual condition.

In some embodiments, the data may include image data generated from an image of the menstrual hygiene product, weight data, time data, resistance data, conductivity data, viscosity data, or biological data of the menstrual fluid.

In some embodiments, the menstrual characteristic may include blood loss, saturation level of the menstrual hygiene product, time that the menstrual hygiene product has been in use, a presence of biomarkers in the menstrual fluid, or blood clot size. In these and other embodiments, blood loss and/or saturation level may be determined based on the weight data and/or the resistance data.

In some embodiments, the menstrual condition may include a blood loss threshold, a saturation threshold, a time threshold, or a presence of a specific biomarker in the menstrual fluid, a blood clot size threshold, or a medication release time.

In some embodiments, the safety action may include providing a notification on a user device and/or at the menstrual hygiene product. In some embodiments, the safety action may include releasing medicine by the menstrual hygiene product.

In some embodiments, the method may further include establishing a baseline for the menstrual characteristic. In these embodiments, the menstrual condition may include a deviation from the baseline for the menstrual characteristic.

In some embodiments, one or more non-transitory computer-readable media may comprise one or more computer-readable instructions, that when executed by one or more processors of a computing device, cause the computing device to perform the method, or part of the method of monitoring menstrual fluid for diagnosis and treatment.

Thus, the embodiments disclosed may improve menstrual hygiene products by allowing information regarding menstruation to be measured by the menstrual hygiene products and monitored in substantially real-time. In addition, the embodiments disclosed may improve women's health by providing and monitoring data captured from menstruation fluids, and performing safety actions based on the data.

The foregoing summary and the following detailed description are explanatory only and are not intended to be in any way limiting. In addition to the illustrative aspects, embodiments, and features described in the present applications, further aspects, embodiments, and features will become apparent by reference to the drawings and the following detailed description.

### BRIEF DESCRIPTION OF THE FIGURES

Example embodiments will be described and explained with additional specificity and detail through the accompanying drawings.

FIG. 1A illustrates a top-view of an example instrument for monitoring menstrual fluid for diagnosis and treatment.

FIG. 1B illustrates a side-view of the example instrument of FIG. 1A.

FIG. 1C illustrates a use-case of the example instrument of FIG. 1A.

FIG. 2 illustrates an example environment that includes an example instrument for monitoring menstrual fluid for diagnosis and treatment.

FIG. 3 illustrates an example menstruation monitoring application.

FIG. 4 illustrates an operational workflow for monitoring menstrual fluid for diagnosis and treatment.

FIG. 5 is a flowchart of a method of monitoring fluid menstrual fluid for diagnosis and treatment.

FIG. 6A illustrates a first simulated experiment of measuring fluid absorption on a menstrual hygiene product utilizing a load cell incorporated in the menstrual hygiene product.

FIG. 6B illustrates a second simulated experiment of measuring fluid absorption on a menstrual hygiene product.

FIG. 6C illustrates a third simulated experiment of measuring fluid absorption on varying sizes of menstrual hygiene products.

FIG. 7A illustrates an example computing system in accordance with one or more embodiments of the present disclosure.

FIG. 7B illustrates another example computing system in accordance with one or

more embodiments of the present disclosure.

FIG. 8A illustrates an embodiment of an example protocol.

FIG. 8B illustrates another embodiment of an example protocol.

FIG. 8C illustrates yet another embodiment of an example protocol.

### DETAILED DESCRIPTION

Menstrual charts and scoring systems to monitor menstrual fluids are often inaccurate because these methods require subjective and qualitative determinations of menstrual characteristics like blood loss, saturation level, and blood clot size. Current menstrual hygiene products only absorb menstrual fluids and do not provide any indication about the health of a woman other than what the woman can visually see on the menstrual hygiene product. Furthermore, current methods of monitoring of menstrual health require either invasive and inconvenient techniques or manual monitoring by the woman.

According to one or more embodiments of the present disclosure, a sensor may be incorporated in a menstrual hygiene product allowing data to be obtained from the menstrual fluid. Based on the data, menstrual characteristics may be determined and monitored for menstrual conditions. Once the menstrual condition is fulfilled, a safety action, which may include notifying the woman or releasing medication, may be performed based on the menstrual condition. Thus, embodiments of the present disclosure provide a woman with information about her health without subjecting her to blood draws or requiring her to track her menstruation daily.

In the following detailed description, reference is made to the accompanying drawings, which form a part hereof. In the drawings, similar symbols typically identify similar components, unless context dictates otherwise. It is to be understood that the figures are diagrammatic and schematic representations of such example embodiments, and are not limiting, nor are they necessarily drawn to scale. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

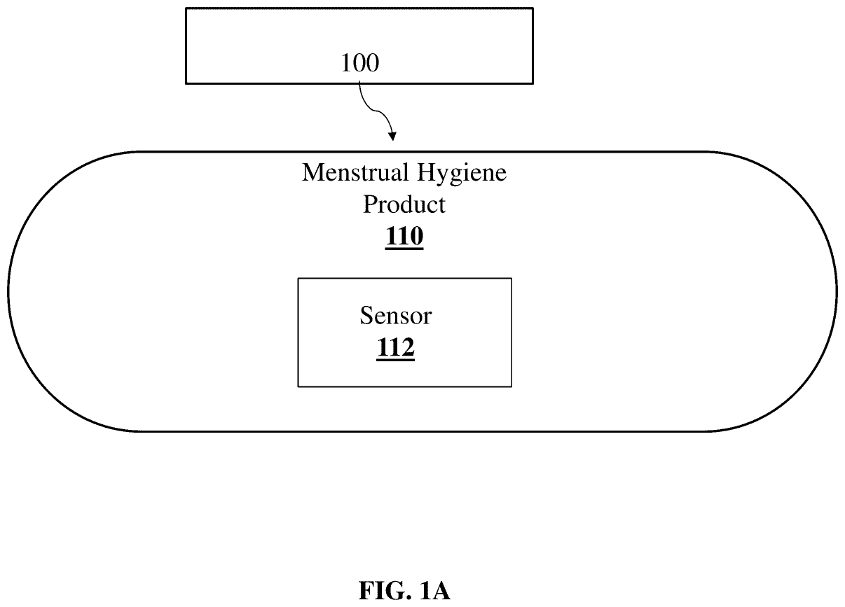

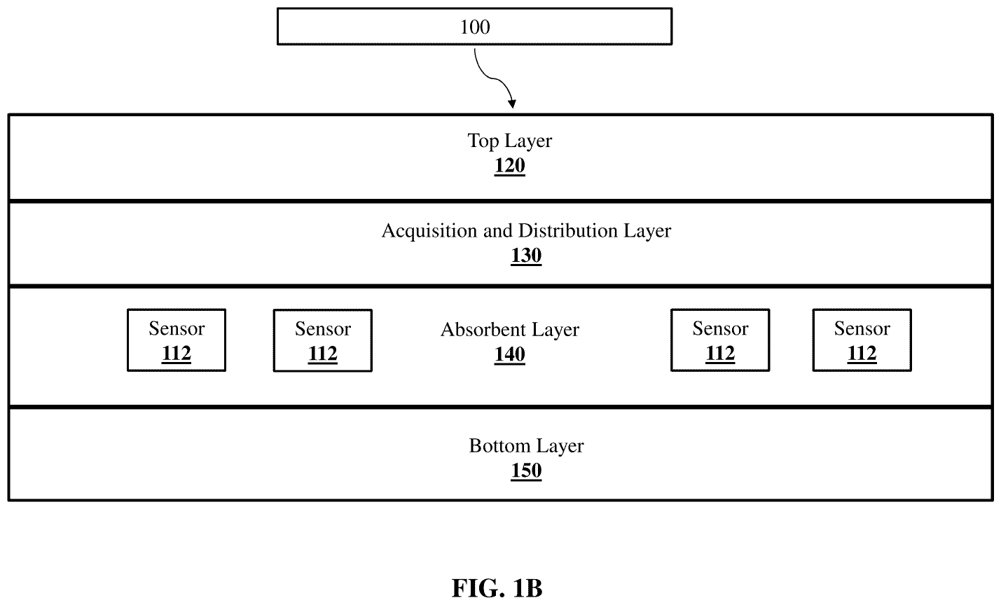

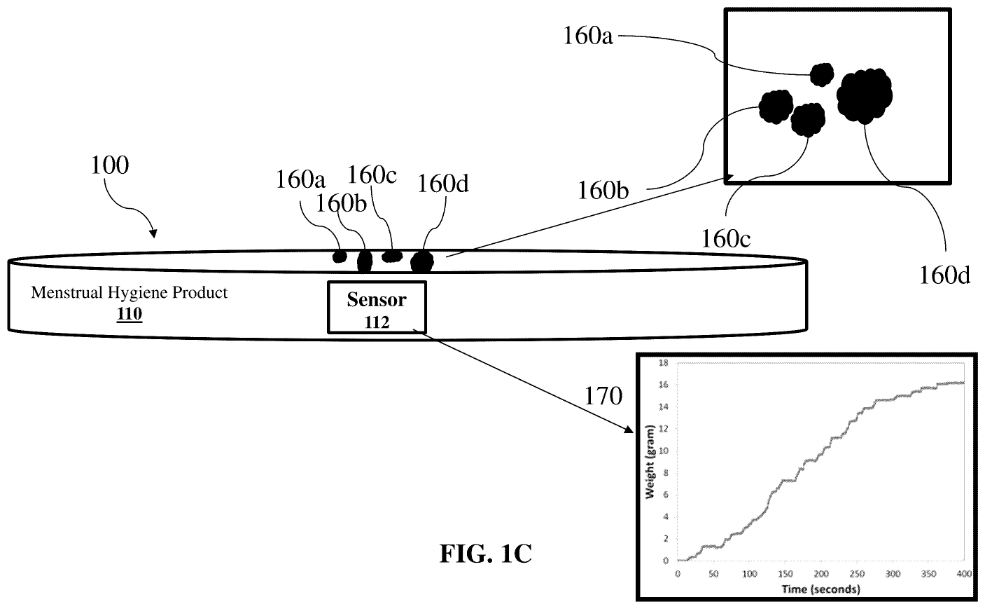

Turning to the figures, FIG. 1A illustrates a top-view of an example instrument 100 for monitoring menstrual fluid for diagnosis and treatment. The example instrument 100 may include a menstrual hygiene product 110 and a sensor 112. As shown in FIG. 1A, the menstrual hygiene product 110 is a sanitary pad, but the menstrual hygiene product 110 may be any menstrual hygiene product. For example, the menstrual hygiene product 110 may be a tampon, a sanitary pad, period underwear, a menstrual cup, a menstrual disc, or a menstrual sponge. In some embodiments, the menstrual hygiene product 110 may include removable layers. In these and other embodiments, the one or more removable layers may analyzed with analysis techniques such as Mass-Spec, DNA sequencing, RNA sequencing, protein identification, cell count, and other molecular characterization techniques.

The sensor 112 may be incorporated in the menstrual hygiene product 112. For example, the sensor 112 may be embedded in one or more layers of a tampon or sanitary pad. In some embodiments, multiple sensors may be incorporated in the menstrual hygiene product 112. For example, two sensors 112 may be embedded in different layers or the same layer of a sanitary pad. In some embodiments, the sensor 112 may be a part of an array of sensors in the menstrual hygiene product 110. For example, an array of sensors 112, as shown in FIG. 1B, may be incorporated into the menstrual hygiene product 110 in order to provide more coverage or redundancy in case of one of the sensors does not function properly. In these and other embodiments, different sensors may detect different attributes of the menstrual fluid.

In some embodiments, the sensor 112 may be a physical sensor, a chemical sensor, a biosensor, an optical sensor, an electrical sensor, an electrochemical sensor, an image sensor, or any other sensor capable of detecting data during menstruation. In some embodiments, the sensor 112 may be a force sensor. For example, the sensor 112 may be a load cell, a weight sensor, a resistive force sensor, or a pressure sensor. In some embodiments, the sensor 112 may be a physiological sensor. For example, the sensor 112 may be a blood-glucose sensor, an oximetry sensor, a temperature sensor, an electrolyte sensor, a lactate sensor, or a heart rate sensor. In embodiments where multiple sensors are incorporated in the menstrual hygiene product 112, the sensors may be any combination of the above-listed sensors and/or any other sensor capable of detecting data during menstruation.

In some embodiments, the sensor 112 may be activated upon coming into contact with menstrual fluids. In some embodiments, the sensors may be powered by batteries. In some of these embodiments, the batteries may be activated by fluid contact. In some of these embodiments, the batteries may be biodegradable.

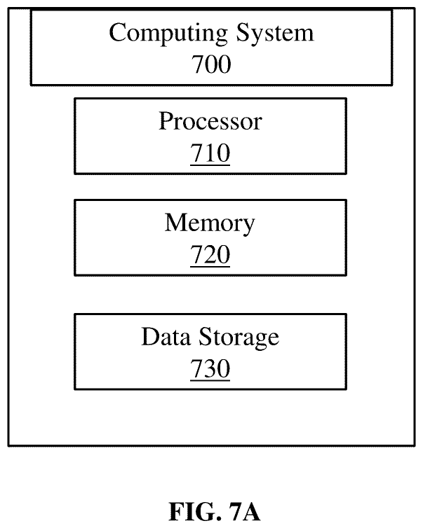

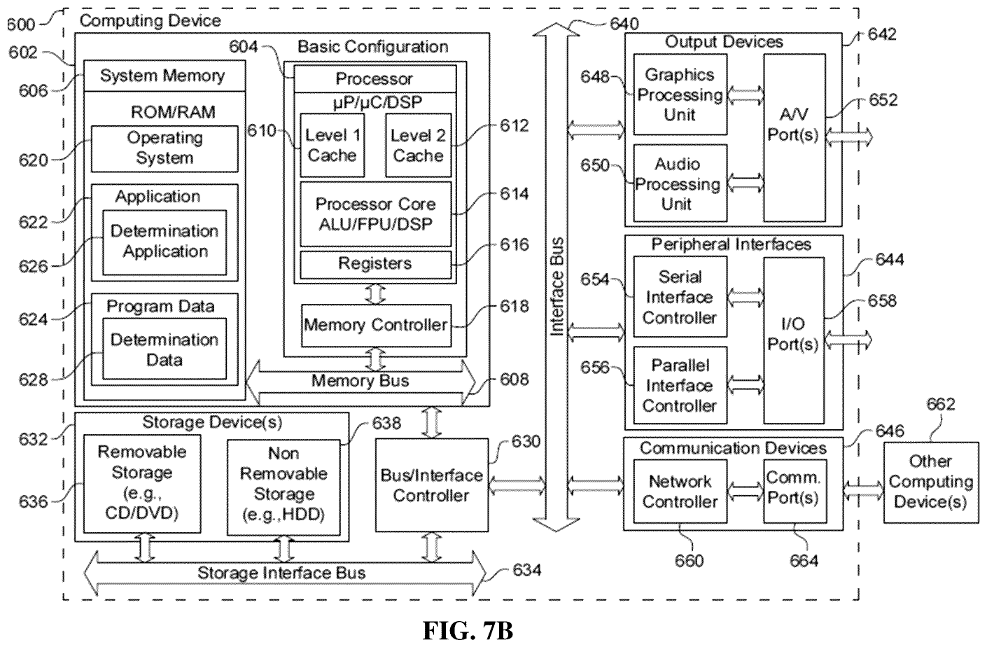

In some embodiments, the instrument 100 may include a processor, a memory, and a storage (See FIG. 7B), which may be incorporated in the menstrual hygiene product 110 and configured to perform operations as described in this disclosure, among other operations. In some embodiments, the instrument 100 may include computer readable-instructions that are configured to be executed by the instrument 100 to perform operations described in this disclosure. The processor may be communicatively coupled to the sensor 112 and the processor may be configured to transmit the data collected by the sensor to a user device (not shown). In some embodiments, the processor may be incorporated into the sensor 112. In general, the processor may include any suitable special-purpose or general-purpose computer, computing entity, or processing device including various computer hardware or software modules and may be configured to execute instructions stored on any applicable computer-readable storage media. For example, the processor may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. The processor may include any number of processors configured to, individually or collectively, perform or direct performance of any number of operations described in the present disclosure. Additionally, one or more of the processors may be present on one or more different electronic devices, such as the user device.

In some embodiments, the instrument 100 may include a camera (not shown) incorporated in the menstrual hygiene product 110. In these embodiments, the camera may capture images of the menstrual fluid contacting the menstrual hygiene product 110. In some embodiments, the processor may be communicatively coupled to the camera and the processor may be configured to transmit images captured by the camera to the user device. In some embodiments, the sensor 112 may be an image sensor and may be used in conjunction with or in replacement of the camera.

The sensor 112 may be configured to collect data from the menstrual fluid contacting the menstrual hygiene product 110 and data concerning attributes of the menstrual hygiene product 110. In these and other embodiments, the sensor 112 may be configured to collect weight data, time data, resistance data, conductivity data, viscosity data, biological data of the menstrual fluid, or image data. The weight data may indicate a weight of the menstrual hygiene product 110, the time data may indicate a length of time that the menstrual hygiene product 110 has been in use, the resistance data may indicate a resistance of the sensor 112, the biological data may indicate the presence of biological molecules in the menstrual fluid, and the image data may indicate blood clot size.

In some embodiments, the data collected by the sensor 112 may be utilized to determine a menstrual characteristic. The menstrual characteristic may include blood loss, saturation level of the menstrual hygiene product, time that the menstrual hygiene product has been in use, a presence of biomarkers in the menstrual fluid, blood clot size, or any other characteristic that may be determined from the menstrual fluid or is associated with menstruation.

In some embodiments, blood loss during menstruation may be determined from the weight data and/or resistance data collected by the sensor 112. For example, the sensor 112 may detect the weight of the menstrual hygiene product 110 or the weight of the menstrual fluid absorbed (for example if the dry weight of the menstrual hygiene product 110 is taken into account), which may increase as the menstrual hygiene product 110 absorbs menstrual fluid. In addition, the sensor 112 may determine the blood loss based on the changes in electrical resistance experienced at the sensor 112 as the menstrual hygiene product 110 absorbs menstrual fluid. For example, the resistance may decrease as the menstrual fluid interacts with the sensor 112, and the resistance changes may be monitored to quantify the amount of blood absorbed by the menstrual hygiene product 110, which may be used to determine blood loss. In some embodiments, the sensor 112 may be capable of differentiating between blood components and other components of the menstrual fluid. In these and other embodiments, the blood loss may be determined based on the weight of the blood components contributing to the weight changes at the sensor 112. In some embodiments, the weight data may be converted to a volume. For example, a known or measured density or specific gravity of blood and/or menstrual fluid may be utilized to convert the weight of menstrual fluid or blood to a volume of menstrual fluid or blood to determine blood loss.

In some embodiments, a saturation level of the menstrual hygiene product 110 may be determined from the weight data and/or the resistance data. In some embodiments, the menstrual hygiene product 110 may have a saturation capacity (or maximum volume the menstrual hygiene product 110 may absorb). In these and other embodiments, the data collected by the sensor 112 may be used to determine how much fluid the menstrual hygiene product 110 has absorbed in comparison to the saturation capacity. For example, the weight data or resistance data collected by the sensor 112 may be converted into a volume of menstrual fluid and that may be compared that to the saturation capacity. Continuing the example, the weight data may indicate an amount of menstrual fluid absorbed (e.g., grams of fluid absorbed) and a known or measured density (e.g. the density of human blood) or specific gravity (e.g., the specific gravity of human blood) may be utilized to convert the weight data into a volume of menstrual fluid or blood absorbed. In some embodiments, the volume of menstrual fluid or blood absorbed may be utilized to determine a saturation level. For example, the volume of menstrual fluid absorbed may be divided by the saturation capacity to determine the saturation level.

In some embodiments, the time data collected by the sensor 112 may be used to determine the time that the menstrual hygiene product has been in use. In these and other embodiments, the sensor 112 may include a timer. In these and other embodiments, the timer may be started when the menstrual hygiene product 110 is put into use. In some embodiments, the timer may be started when the sensor detects weight changes. For example, once the sensor 112 indicates that the menstrual hygiene product 110 has begun to absorb menstrual fluid the timer may be started. Additionally or alternatively, the instrument 100 may be communicatively coupled with a user device and the timer may begin upon communication being established with the user device.

In some embodiments, biological data collected by the sensor 112 may be utilized to determine a presence of biomarkers in the menstrual fluid. In some embodiments, the biological data may include biomolecule concentrations (e.g., metabolite concentrations, hormone levels, protein concentrations), physiological parameters (e.g., pH levels, temperature, oxygen saturation), electrochemical signals (e.g. ion concentrations, redox potential). For example, the biological data collected by the sensor may be used to determine the presence of disease-based biomarkers such as those for diabetes, cancer, sexually transmitted diseases, and anemia.

In some embodiments, the image data collected by the sensor 112 and/or the camera may be utilized to determine blood clot size. The processor may perform image processing to determine the size and/or number of blood clots captured in the image data by the sensor 112 and/or the camera. For example, the processor may determine the size of blood clots captured in the image based on quantitative metrics like surface area or volume that the blood clots take up on the menstrual hygiene product, and/or based on qualitative metrics (e.g. grape size or strawberry size). Additionally or alternatively, the processor may group the blood clots into size groupings based on quantitative and/or qualitative metrics.

In some embodiments, the menstrual characteristic may be monitored by the processor for a menstrual condition. In these and other embodiments, the menstrual condition may include a blood loss threshold, a saturation threshold, a time threshold, a presence of specific biomarkers, a blood clot size threshold, or a medication release time.

In embodiments where the menstrual condition includes a blood loss threshold, the blood loss threshold may be a specific amount of blood loss based on the volume or the weight of the blood lost while the menstrual hygiene product 110 is in place. Thus, the menstrual condition (blood loss threshold) may be fulfilled when the blood lost meets and/or exceeds the specific amount of blood loss.

In some embodiments, the blood loss threshold may be cumulative over the number of menstrual hygiene products 110 used and/or the blood loss threshold may be set for each menstrual hygiene product 110 used by a user. In some embodiments, the blood loss threshold may indicate that the menstrual hygiene product 110 may no longer be capable of holding fluid and/or may indicate that the menstrual hygiene product 110 has been in place long enough to warrant concern for toxic shock syndrome. In some embodiments, the blood loss threshold may be up to 1 g of blood, up to 2 g of blood, up to 3 g of blood, up to 4 g of blood, up to 5 g of blood, up to 10 g of blood, up to 20 g of blood, up to 30 g of blood, or up to about 40 g of blood or any other blood loss threshold within the absorbency capacity of the menstrual hygiene product 110. Additionally or alternatively, the blood loss threshold may be up to 1 ml of blood, up to 2 mL of blood, up to 3 mL of blood, up to 4 mL of blood, up to 5 mL of blood, up to 10 mL of blood, up to 20 mL of blood, up to 30 mL of blood, or up to up to 40 mL of blood or any other blood loss threshold within the absorbency capacity of the menstrual hygiene product 110. While expressed in terms of g and/or mL, the blood loss threshold may be determined based on any measurement of blood loss. In some embodiments, there may be multiple blood loss thresholds. For example, a first blood loss threshold may be set at 10 mL of blood loss or menstrual fluid loss and a second blood loss threshold may be set at 15 mL of blood loss or menstrual fluid loss.

In embodiments where the menstrual condition includes a saturation threshold, the saturation threshold may be a specific saturation based on the volume or the weight of the blood lost compared to the saturation capacity of the menstrual hygiene product 110. Thus, the menstrual condition (saturation threshold) may be fulfilled when the saturation level meets and/or exceeds the specific saturation.

In some embodiments, the saturation threshold may indicate that the menstrual hygiene product 110 may no longer be capable of holding fluid or may indicate that the menstrual hygiene product 110 has been in place long enough to warrant concern for toxic shock syndrome. In some embodiments, the saturation threshold may be up to 10% of saturation capacity, up to 20% of saturation capacity, up to 30% of saturation capacity, up to 40% of saturation capacity, up to 50% of saturation capacity, up to 60% of saturation capacity, up to 70% of saturation capacity, up to 80% of saturation capacity, up to 90% of saturation capacity, up to 100% of saturation capacity or any other saturation capacity within the saturation capacity of the menstrual hygiene product 110. While expressed in terms of percentage, the saturation threshold may be determined based on any measurement of saturation level (e.g. fractions, decimals, etc.). In some embodiments, there may be multiple saturation thresholds. For example, a first saturation threshold may be set at 70% of saturation capacity and a second saturation threshold may be set at 100% of saturation capacity.

In embodiments where the menstrual condition includes a time threshold, the time threshold may be a specific amount of time. In some embodiments, the time threshold may be a specific amount of time that the menstrual hygiene product 110 has been in place. Additionally or alternatively, the time threshold may be a medication release time. Thus, the menstrual condition (time threshold) may be fulfilled when, for example, the time that the menstrual hygiene product 110 has been in place exceeds the time threshold.

In some embodiments, the time threshold may indicate that the menstrual hygiene product 110 may no longer be capable of holding fluid or may indicate that the menstrual hygiene product 110 has been in place long enough to warrant concern for toxic shock syndrome. For example, toxic shock syndrome may occur when a tampon has been left in for between 4-8 hours or longer. Continuing the example, the time threshold may be set at 1 hour, 2 hour, 3 hours, 4 hours, 5 hours, 6 hours, 7 hours, or 8 hours.

While expressed in terms of hours, the time threshold may be determined based on any unit of measurement of time (e.g. minutes, seconds). In some embodiments, there may be multiple time thresholds. For example, a first time threshold may be set at 3 hours, a second time threshold at 4 hours, and a third time threshold at 8 hours.

In embodiments where the menstrual condition includes a presence of specific biomarkers, the menstrual condition may be fulfilled when the sensor 112 detects the presence of a specific biomarker. In some embodiments, the specific biomarker may include detection of pathogens, levels of biological molecules, or disease-based biomarkers such as those for diabetes, cancer, sexually transmitted diseases, and anemia. For example, the presence of HIV antibodies may indicate an HIV infection. In another example, the sensor 112 may detect the presence of Candida antigen or Candida metabolites in the menstrual fluid, which may indicate a yeast infection. Thus, a known biomarker can be assayed for on the product for detection of a disease biomarker and pathogen biomarkers, or biomarker panels for biological signatures.

In embodiments where the menstrual condition includes a blood clot size threshold, the blood clot size threshold may be quantitative (e.g., volume or surface area of blood clot) or qualitative. In some embodiments, the menstrual condition may be fulfilled when the image data collected by the sensor 112 indicates a blood clot meeting or exceeding the blood clot size threshold. For example, the image data collected by the sensor 112 may indicate that a blood clot is larger than the size of a grape, the blood clot size threshold may be grape size, and thus the menstrual condition may be fulfilled. On the other hand, if the blood clot size is less than that of a grape the menstrual condition may not be fulfilled. In another example, the image data collected by the sensor 112 may indicate that the surface area of a blood clot is 1.5 square inches, the blood clot size threshold may be 1.3 square inches, and thus the menstrual condition may be fulfilled.

In some embodiments, the blood clot size threshold may be up to 0.9 square inches, up to 1.0 square inches, up to 1.1 square inches, up to 1.2 square inches, up to 1.3 square inches, up to 1.5 square inches, up to 1.75 square inches, or up to 2 square inches or any other blood clot size threshold. While expressed in terms of square inches, the blood clot size threshold may be determined based on any other unit of measurement or any other measurement of blood clot size. In some embodiments, there may be multiple blood clot size thresholds. For example, a first blood clot size threshold may be fulfilled upon detection of any blood clot and a second blood clot size threshold may be fulfilled upon a blood clot size exceeding 1.5 square inches.

In some embodiments, the processor may establish a baseline for the menstrual characteristic based on the data collected from the sensor 112. In these and other embodiments, the instrument 100 may account for historical data regarding the menstrual characteristic. In some embodiments, the baseline may be a range of the menstrual characteristic, an average value of the menstrual characteristic, a minimum value of the menstrual characteristic, a maximum value of the menstrual characteristic, a specific value of the menstrual characteristic, a qualitative metric of the menstrual characteristic (e.g., absence or presence of biomarkers or qualitative blood clot size), or any other reference measurement which future determinations of menstrual characteristics may be compared to. For example, the baseline may be the maximum blood loss of a user based on previous blood loss data, the baseline may be average blood loss data of a user based on previous blood loss data, or the baseline may be the range from minimum to maximum blood loss based on previous blood loss data.

In these and other embodiments, the menstrual condition may be a deviation from the baseline for the menstrual characteristic. For example, the menstrual condition may be the blood loss falling outside of a baseline range of blood loss. In some embodiments, the baseline may be established from a first use of the instrument 100. In some embodiments, the baseline may be established from uses of multiple instruments 100. In some embodiments, the baseline may be established from user data obtained from medical records, medical reports, lab results, patient portals or the like.

In some embodiments, a safety action may be performed by the instrument 100 and/or a user device based on fulfillment of the menstrual condition. In some embodiments, the processor, which may be included in the instrument 100 and/or the sensor 112, may determine that the menstrual condition has been fulfilled. In some embodiments, the safety action may include providing a notification to a user device or the instrument 100. In these and other embodiments, the instrument 100 could be communicatively coupled with a user device. In some of these embodiments, the instrument 100 may send a notification signal to the user device and upon receiving the signal the user device may notify the user audibly, visually, and/or haptically that the menstrual condition has been fulfilled. For example, a time threshold of 4 hours may be exceeded and the instrument 100 may provide a notification to a user device that the time threshold has been exceeded and warning the user of the potential for toxic shock syndrome. In some embodiments, the instrument 100 may provide audible (e.g. a ding), visual (e.g. a light included in the instrument blinking), or haptic feedback (e.g. the instrument vibrating) that the menstrual condition has been fulfilled.

In some embodiments, the safety action may include releasing medicine from the menstrual hygiene product 110. For example, a suppository for treating a yeast infection may be included in a core of a tampon. The menstrual condition may be the presence of Candida, and determining that the data collected by the sensor 112 indicates the presence of Candida (fulfillment of menstrual condition), the instrument 100 may release the suppository. In some embodiments, the medicine may be released upon a signal being received at the processor to dispense the medicine. For example, the processor may receive a signal from the user device that the menstrual condition of the presence of Candida has been fulfilled and the processor may in turn cause the medicine to be released from the core of the tampon. In some embodiments, the medicine may be released based on the processor receiving data from the sensor indicating that the menstrual condition has been fulfilled. For example, the processor may determine that the menstrual condition of the presence of Candida has been fulfilled and the processor may in turn cause the medicine to be released from the core of the tampon. In some embodiments, the medicine may be released upon the sensor contacting menstrual fluid. For example, the sensor may be activated by contacting menstrual fluid and upon contact, the processor may cause the medicine to be released from the tampon.

In some embodiments, the menstrual hygiene product 110 may be constructed from biodegradable components. For example, the menstrual hygiene product 110 may be constructed from cotton, coconut, banana fibers, bamboo, coconut fibers, any other biodegradable component or any combination of biodegradable components. In some embodiments, the menstrual hygiene product 110 may be constructed from non-biodegradable components. For example, the menstrual hygiene product 110 may be constructed from synthetic materials like polyester or rayon.

In some embodiments, the sensor may be made of natural and synthetic biodegradable components. For example, the sensor may be made of polylactic acid (PLA), poly(lactic-co-glycolic acid) (PLGA), poly(L-lactic acid) (PLLA), poly-γ-glutamic acid (PGA), polycaprolactone (PCL), plant-based polysaccharides (e.g., pectin, cellulose, alginate, dextran), or animal-derived materials (e.g., collagen, chitin, chitosan, shellac, gelatin, silk, chitosan).

In some embodiments, the instrument 100 may be capable of communicatively coupling with a user device. For example, the instrument 100 may be capable of communicatively coupling with cell phones, laptops, computers, or other compatible devices. In these and other embodiments, the instrument 100 may be communicatively coupled with the user device via a Personal Area Network (PAN), a Local Area Network (LAN), a Metropolitan Area Network (MAN), a Wide Area Network (WAN), a Storage Area Network (SAN), a cellular network, the Internet, an optical network, Wi-Fi, Bluetooth, a Zigbee connection, a cellular network connection, a thread connection, a Z-wave connection, or other similar communication modalities. In these and other embodiments, the instrument 100 may transmit the data collected by the sensor to the user device and/or the instrument 100 may notify the user device that a menstrual condition has been fulfilled. For example, the instrument 100 may transmit the weight data to the user device and/or the instrument 100 may notify that user device that the saturation threshold has been exceeded indicating that the menstrual hygiene product 110 should be replaced. In these and other embodiments, the data collected by the sensor 112 may be transmitted to the user device in substantially real-time.

FIG. 1B illustrates a side-view of the example instrument 100 of FIG. 1A. As shown in FIG. 1B, the menstrual hygiene product 110 is a sanitary pad, but the menstrual hygiene product 110 may be any menstrual hygiene product. FIG. 1B illustrates the sanitary pad having one or more layers. The one or more layers may include a top layer 120, an acquisition and distribution layer 130, an absorbent layer 140, and a bottom layer 150.

The top layer 120 may be a top-sheet that allows the menstrual fluid to enter the menstrual hygiene product 100 and wicks away the menstrual fluid from the skin to keep the surface of the skin relatively dry. The acquisition and distribution layer 130 may distribute the menstrual fluid along the length of the menstrual hygiene product 110 once the menstrual fluid enters the menstrual hygiene product 110. Thus, the acquisition and distribution layer 130 may prevent the menstrual fluid from pooling in one spot on the menstrual hygiene product 110. The absorbent layer 140 may be an absorbent core, which may absorb and contain the menstrual fluid once the menstrual fluid has been acquired and distributed by the acquisition and distribution layer 130. The bottom layer 150 provides a relatively waterproof barrier to prevent the menstrual hygiene product 110 from leaking. The underwear-facing side of the bottom layer 150 may include an adhesive to secure the pad to the underwear.

As shown in FIG. 1B, the sensor 112 may be incorporated in the absorbent layer 140 of the menstrual hygiene product 110. In some embodiments, multiple sensors 112 may be incorporated in the menstrual hygiene product 110. For example, one sensor may be incorporated in the absorbent layer 112 and one sensor may be incorporated into the acquisition and distribution layer 130.

The instrument 100 is illustrated as including a menstrual hygiene product 110 with the top layer 120, the acquisition and distribution layer 130, the absorbent layer 140, and the bottom layer 150 for purposes of exemplifying the incorporation of the sensor 112 into a specific type of sanitary pad. The menstrual hygiene product 110 may not include some or any of these layers and the sensor 112 may still be incorporated into the menstrual hygiene product 110.

FIG. 1C illustrates an example use-case of the example instrument 100 of FIG. 1A. As shown in FIG. 1C, menstrual fluid may contact the menstrual hygiene product 110. The sensor 112 may obtain data from the menstrual fluid. As shown in FIG. 1C, the sensor data includes weight data, time data, and image data. In this use case, multiple sensors may be incorporated in the menstrual hygiene product 110. For example, an image sensor may be incorporated to collect the image data, a load cell may be incorporated to collect the weight data, and a timer may be incorporated in one or both of the sensors or the menstrual hygiene product 110 to collect time data.

The data may be processed by the processor (not shown) to determine a menstrual characteristic based on the data and the processor may monitor the menstrual characteristic for a menstrual condition. As shown in FIG. 1C, there are blood clots 160a-d on the menstrual hygiene product 110. The sensor 112 may collect image data of the blood clots 160a-d, and the processor may determine the size of the blood clots 160a-d. The processor may monitor the size of the blood clots 160a-d to determine if any of the blood clots meet a blood clot size threshold. For example, the blood clots 160a-c may not meet the blood clot size threshold, but the blood clot 160d may meet or exceed the blood clot size threshold. The instrument 100 and/or a user device communicatively coupled with the instrument 100 may perform a safety action based on the blood clot 160d exceeding the blood clot size threshold. For example, the instrument 100 may transmit a notification to the user device that the blood clot size threshold has been exceeded.

As shown in FIG. 1C, the sensor 112 may collect weight data and time data as illustrated by the weight-time chart 170. The weight-time chart 170 illustrates that the sensor 112, as shown in FIG. 1C, is activated by menstrual fluid contacting the menstrual hygiene product 110 and, consequently, the weight of the menstrual hygiene product 110 increases as it absorbs the menstrual fluid. The processor may determine how long the menstrual hygiene product 110 has been in use based on the time data and may monitor the time in use for a time threshold. The processor may determine blood loss based on the weight data and may monitor the blood loss for a blood loss threshold. For example, the weight-time chart 170 depicts that the weight of menstrual fluid is about 16 g after 400 seconds. For the purposes of illustration, the weight of the menstrual fluid may be divided by the known or measured density of menstrual blood, which may be 1.06 g/mL to determine a volume of blood loss. In this example, the calculated volume of blood loss would be 15.09 mL.

The instrument 100 or a user device communicatively coupled with the instrument 100 may perform a safety action based on blood loss exceeding the blood loss threshold. Continuing the example in the preceding paragraph, if the blood loss threshold was 15 mL, the instrument 100 may transmit a notification to the user device that the blood loss threshold has been exceeded because the calculated volume of blood loss at 15.09 mL exceeds the blood loss threshold.

Modifications, additions, or omissions may be made to the instrument 100 without departing from the scope of the present disclosure. For example, in some embodiments, the instrument 100 may not include a processor and a user device may receive the data collected by the instrument 100 and process the data remotely. In another example, the sensor 112 may have processing capabilities or an internal processor or the processor may be separate from the sensor 112.

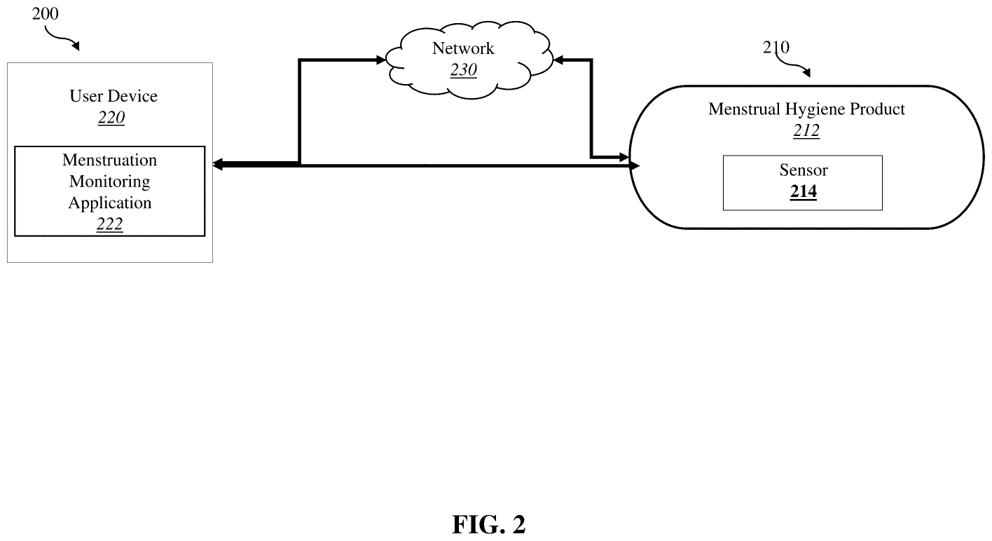

FIG. 2 illustrates an example environment 200 for monitoring menstrual fluid for diagnosis and treatment. The example environment 200 may include an instrument 210, a user device 220, and a network 230. The instrument 210 may include a menstrual hygiene product 212 and a sensor 214. The instrument 210, the menstrual hygiene product 212, and the sensor 214 may all be similar to and perform similar functions as the instrument 100, the menstrual hygiene product 110, and the sensor 112 described with reference to FIG. 1.

In some embodiments, the network 230 may be configured to communicatively couple the user device 220 and the instrument 210. In some embodiments, the network 230 may be any wired or wireless network, or combination of multiple networks, configured to send and receive communications between systems and devices. In some embodiments, the network 110 may include a Personal Area Network (PAN), a Local Area Network (LAN), a Metropolitan Area Network (MAN), a Wide Area Network (WAN), a Storage Area Network (SAN), a cellular network, a mesh network the Internet, an optical network, or some combination thereof. In these and other embodiments, the instrument 210 may transmit the data collected by the sensor to the user device 220 via the network 230.

In some embodiments, the user device 220 and the instrument 210 may be communicatively coupled via Bluetooth, Zigbee connection, a thread connection, a Z-wave connection, or other similar communication modalities. In these and other embodiments, the instrument 210 may transmit the data collected by the sensor directly to the user device 220 and/or the instrument 210 may notify the user device that a menstrual condition has been fulfilled.

In some embodiments, the user device 220 may be any computer system capable of communicating over the network 230, capable of communicating with the instrument 210, and/or capable of monitoring menstrual characteristics for menstrual conditions. For example, the user device 220 may be a desktop computer, a laptop computer, a smartphone, a mobile phone, a tablet computer, or any other computing device that may be used for communication between devices over the network 230 and capable of monitoring characteristics for menstrual conditions.

In some embodiments, the user device 220 and/or the instrument 210 may include memory, storage, and/or at least one processor, which are configured to perform operations as described in this disclosure, among other operations. In some embodiments, the user device 220 and/or the instrument 210 may include computer-readable instructions that are configured to be executed by the user device 220 and/or the device 210, respectively, to perform operations described in this disclosure.

The user device 220 includes a menstruation monitoring application 222. To monitor menstrual fluid for diagnosis and treatment, the menstruation monitoring application 222 may obtain data from the instrument 210. For example, the menstruation monitoring application 222 may obtain weight data, time data, image data, resistance data, conductivity data, viscosity data, or biological data from the instrument 210. In some embodiments, the data obtained may be processed and in some embodiments the data may be raw data from the sensor 214.

In some embodiments, the menstruation monitoring application 222 may obtain data from the user device 220. For example, the user device 220 may include a camera and the user may capture an image of the menstrual hygiene product 212 for the menstruation monitoring application to determine, for example, blood clot size.

Once this data has been obtained, the menstruation monitoring application 222 may determine a menstrual characteristic based on the data from the instrument 210. In some embodiments, the menstruation monitoring application 222 may determine menstrual characteristics including blood loss, saturation level of the menstrual hygiene product 212, time that the menstrual hygiene product 212 has been in use, a presence of biomarkers in the menstrual fluid, or blood clot size. For example, the menstruation monitoring application 222 may determine blood loss from weight data and/or the resistance data measured by the sensor 214. In some embodiments, the user of the user device 220 may select the specific menstrual characteristics that the menstruation monitoring application 222 may determine. In some embodiments, the menstruation monitoring application 222 may determine multiple menstrual characteristics.

In some embodiments, the menstruation application module 222 may determine a baseline for the menstrual characteristic based on the data the menstruation monitoring application 222 receives from the instrument 210. The baseline may be determined based on current and/or past data received from the instrument 210. In some embodiments, the baseline may be a range of the menstrual characteristic, an average value of the menstrual characteristic, a minimum value of the menstrual characteristic, a maximum value of the menstrual characteristic, a specific value of the menstrual characteristic, a qualitative metric of the menstrual characteristic (e.g., absence or presence of biomarkers or qualitative blood clot size), or any other reference measurement which future determinations of menstrual characteristics may be compared to.

In some embodiments, the baseline may be established from a first use of the instrument 210. In some embodiments, the baseline may be established from uses of multiple instruments 210. In some embodiments, the baseline may be established based on user data obtained by the menstruation monitoring application 222 from medical records, medical reports, lab results, patient portals or other sources of user data.

In some embodiments, the menstrual condition may include a deviation from the baseline for the menstrual characteristic. For example, the baseline may be that there is no presence of Candida has been detected, and the deviation may be a menstrual characteristic showing the presence of Candida in the menstrual fluid. In some embodiments, the user of the user device 220 or the user of a different device (not shown) may manually set the deviation from the baseline for the menstrual characteristic.

Once the menstrual characteristic has been determined, the menstruation monitoring application 222 may monitor the menstrual characteristic for a menstrual condition. In some embodiments, the menstrual condition may include a blood loss threshold, a saturation threshold, a time threshold, a presence of a specific biomarker in the menstrual fluid, a blood clot size threshold, or a medication release time. For example, the menstruation monitoring application 222 may monitor the biological data of the menstrual fluid to determine if a specific biomarker like HIV antibodies are present in the menstrual fluid.

Once the menstrual condition has been fulfilled, the menstruation monitoring application 222 may perform a safety action based on the fulfillment of the menstrual condition. In some embodiments, the safety action may include providing a notification on the user device 220. In some embodiments, the notification may be visual, audible, and/or haptic. For example, the menstruation monitoring application 222 may determine that the time that the menstrual hygiene product 212 has been in use has exceeded a time threshold, and the menstruation monitoring application 222 may provide a notification on the user device 220 indicating that the time threshold has been exceeded. In some embodiments, the user device 220 may include a display and the menstruation monitoring application 222 may cause a visual notification, for example, a push notification to appear on the display of the user device 220 indicating that the time threshold has been exceeded. In these and other embodiments, the menstruation monitoring application 222 may provide a notification on the user device via email, text, phone call, or other communication methods. In some embodiments, the notification provided on the user device 220 may be a haptic notification, for example vibration. In some embodiments, the notification provided on the user device 220 may be an audible notification like a ring. Thus, the menstruation monitoring application 222 may prevent the user from experiencing toxic shock syndrome if the menstrual hygiene product 212 is in use for too long.

Additionally or alternatively, the safety action may include providing a notification to the instrument 210. In some embodiments, the notification may be visual, audible, and/or haptic. For example, the menstruation monitoring application 222 may determine that the time that the menstrual hygiene product 212 has been in use has exceeded a time threshold, and the menstruation monitoring application 222 may cause a notification to be provided on the instrument 210 indicating that the time threshold has been exceeded. For example, the menstruation monitoring application 222 may send a signal via the network 230 or directly to the instrument 210 to provide a notification on the instrument 210. For example, the instrument 210 may vibrate, the sensor 214 may include an indicator that, for example, starts to blink red, or the instrument 210 may emit an audible sound.

Additionally or alternatively, the safety action may include causing the release of medicine at the menstrual hygiene product 212. For example, once the menstrual condition has been fulfilled the menstruation monitoring application 222 may send a signal via the network 230 or directly to the instrument 210 to release medicine at the menstrual hygiene product 212. For example, a suppository for treating a yeast infection may be included in a core of a tampon. The menstrual condition may be the presence of Candida, and, once the data collected by the sensor 214 indicates the presence of Candida (fulfillment of menstrual condition), the instrument 210 may release the suppository. In some embodiments, the menstrual condition may be a medicine release time, and, once the menstruation monitoring application 222 determines that the time data meets or exceeds the medicine release time, the menstruation monitoring application may cause the instrument 210 to release medicine included in the menstrual hygiene product 212. In some embodiments, the medicine may be released upon the sensor 214 contacting menstrual fluid. For example, the sensor 214 may be activated by contacting menstrual fluid and once fluid contacts the sensor, the instrument 210 may cause the medicine to be released from the tampon.

In some embodiments, the menstruation monitoring application 222 may build a history or a user profile of the user of the instrument 210. For example, the menstruation monitoring application 222 may store the data received from the instrument 210 in a memory of the user device 220. The menstruation monitoring application 222 may use the data stored to help determine the baseline for the menstrual characteristics, and the menstrual conditions may be based on the baseline. In some embodiments, the user profile or user history may be accessible to the user on the user device 220 to track the menstrual characteristics of the user.

Modifications, additions, or omissions may be made to the environment 200 without departing from the scope of the present disclosure. For example, in some embodiments, the user device 220 may not be included and the operations described may be performed by the instrument 210. In other embodiments, multiple user devices 220 may be included and one or more of the user devices 220 may include the menstruation monitoring application 222. For example, the instrument 210 may be communicatively coupled with a user device 220 that belongs to the user of the instrument 210 and the instrument 210 may also be communicatively coupled with another user device 220 that belongs to a medical professional, a parent of the user, etc. which also allows those users to monitor the menstrual health of the individual using the instrument 210. In some embodiments, the data obtained by the sensor 214 may be processed by a device separate from the user device 220 before the data is transmitted to the user device 220. In some embodiments, the operations and processes described in the disclosure may be performed by the instrument 210 and the user device 220 for redundancy.

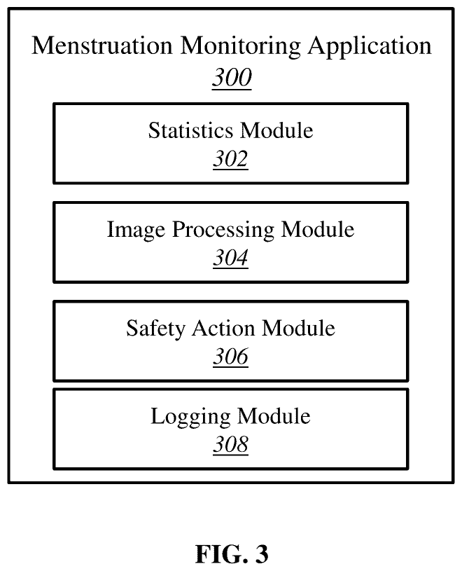

FIG. 3 illustrates an example menstruation monitoring application 300. The menstruation monitoring application 300 may be similar to and perform similar functions as the menstruation monitoring application 222 described with reference to FIG. 2. The menstruation monitoring application 300 may include a statistics module 302, an image processing module 304, a safety action module 306, and a logging module 308. Each of the statistics module 302, image processing module 304, safety action module 306, and logging module 308 may communicatively coupled with any or all of the other modules included in the menstruation monitoring application 300.

In some embodiments, the menstruation monitoring application 300 may be included in a user device. In these embodiments, the user device may be a desktop computer, a laptop computer, a smartphone, a mobile phone, a tablet computer, or any other computing device. The user device may be similar to and perform similar functions as the user device 220 described with reference to FIG. 2.

The statistics module 302 may obtain data from an instrument including a menstrual hygiene product that incorporates a sensor. The instrument may be similar to and perform similar functions as the instrument 100 and the instrument 210 of FIGS. 1 and 2, respectively. The statistics module 302 may obtain the data via a network. The network may be similar to and perform similar functions as the network 230 described with reference to FIG. 2.

In some embodiments, the statistics module 302 may obtain raw data from the instrument and/or the statistics module 302 may obtain processed data from the instrument. In some embodiments, the data may include weight data, time data, resistance data, conductivity data, viscosity data, and/or biological data of the menstrual fluid. In some embodiments, the statistics module 302 may obtain both raw data and processed data from the instrument. In these embodiments, the statistics module 302 may process the raw data and compare the processed data of the statistics module 302 against the processed data received from the instrument.

In some embodiments, the statistics module 302 may determine a menstrual characteristic from the data. The menstrual characteristic(s) may be similar to those described with reference to FIG. 1 and FIG. 2. For example, the statistics module 302 may determine a volume of blood loss from the weight and/or resistance data received from the instrument. In some embodiments, the statistics module 302 may monitor the menstrual characteristic for a menstrual condition. The menstrual condition(s) may be similar to those described with reference to FIG. 1 and FIG. 2.

In some embodiments, the statistics module 302 may establish a baseline for the menstrual characteristic. The baseline may be similar to the baselines described with reference to FIG. 1 and FIG. 2. In some embodiments, the menstrual condition may include a deviation from the baseline for the menstrual characteristic.

In some embodiments, the image processing module 304 may obtain image data from the instrument and/or the user device and process the data. The image data may be generated from an image of the menstrual hygiene product. In some embodiments, the image data obtained may be raw data and/or processed data. In some embodiments, the image data may be processed by a processor included in the user device in which the menstruation monitoring application 300 may be included. In some embodiments, the statistics module 302 may receive the image data and send the image data (raw or processed) to the image processing module 304.

In some embodiments, the image processing module 304 may determine a menstrual characteristic based on the image data received. The menstrual characteristic(s) may be similar to the menstrual characteristic(s) described with reference to FIG. 1 and FIG. 2. For example, the image processing module 304 may determine blood clot size based on the image data. In some embodiments, the user device, of which the menstruation application module 300 may be a part, may include an image processor through which the image processing module 304 may process the image data. In some embodiments, the image processing module 304 may monitor the menstrual characteristic for a menstrual condition. The menstrual condition(s) may be similar to those described with reference to FIG. 1 and FIG. 2.

In some embodiments, the menstruation monitoring application may include a safety action module 306. In some embodiments, the safety action module 306 may perform or cause performance of a safety action based on fulfillment of the menstrual condition. In some embodiments, the safety action module 306 may provide a notification on the user device of which the menstruation monitoring application 300 may be a part. In some embodiments, the menstruation monitoring application 300 may cause a notification to be provided on the instrument. In some embodiments, the menstruation monitoring application 300 may cause medication to be released at the instrument.

In some embodiments, the logging module 308 may log the data which may be received by the statistics module 302 and/or the image processing module 304. In these and other embodiments, the logging module 308 may log the menstrual characteristics determined by the statistics module 302 and/or the image processing module 304. For example, the logging module 308 may store and provide a daily log of blood loss and blood clot size. Additionally or alternatively, the logging module 308 may log the menstrual conditions fulfilled and/or compare the menstrual conditions to the menstrual characteristics. Additionally or alternatively, the logging module 308 may log the safety actions performed. In some embodiments, the logging module 308 may store and provide historical data of menstrual characteristics of the user of the instrument.

Modifications, additions, or omissions may be made to the menstruation monitoring application 300 without departing from the scope of the present disclosure. In some embodiments, the operations and processes described above may be performed by any of the modules described or additional modules may be added that perform these operations and processes. For example, the statistics module 302 may perform the operations of the image processing module 304. In another example, the logging module may establish the baseline for the menstrual characteristics.

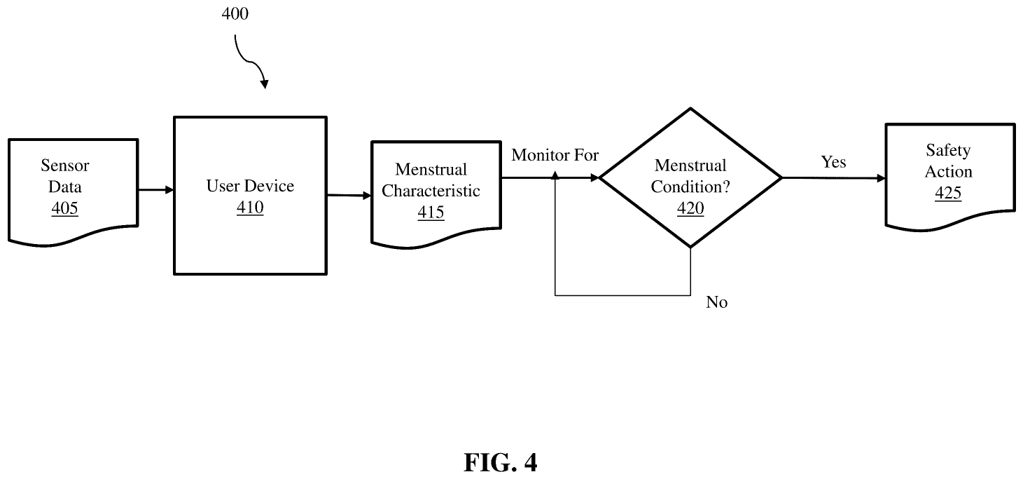

FIG. 4 illustrates an operational workflow 400 for monitoring menstrual fluid for diagnosis and treatment. As shown in FIG. 4, sensor data 405 may be obtained by a user device 410. In some embodiments, the sensor data 405 may be raw. In some embodiments, the sensor data 405 may be processed. The sensor data 405 may be data captured by a sensor that may be similar to and perform similar functions as the sensors 112 and 214 described with reference to FIG. 1 and FIG. 2, respectively. In some embodiments, the user device 410 may be similar to and perform similar functions as the user device 220 described with reference to FIG. 2. In some embodiments, the user device may be similar to and perform similar functions as the instruments 100 and 210 of FIGS. 1 and 2, respectively.

The user device 410 may determine a menstrual characteristic 415 from the sensor data 405. The menstrual characteristic(s) 415 determined may include blood loss, saturation level of a menstrual hygiene product, time that the menstrual hygiene product has been in use, a presence of biomarkers in the menstrual fluid, or blood clot size. The menstrual characteristic(s) 415 may be similar to those described previously.

The user device 410 may monitor the menstrual characteristic 415 for a menstrual condition 420. Once the user device 410 determines that the menstrual condition has been fulfilled, a safety action 425 may be performed. If the user device 410 does not determine that the menstrual condition has been fulfilled, the user device 410 may continue monitoring the menstrual characteristics 415 determined as the sensor data 405 is obtained.

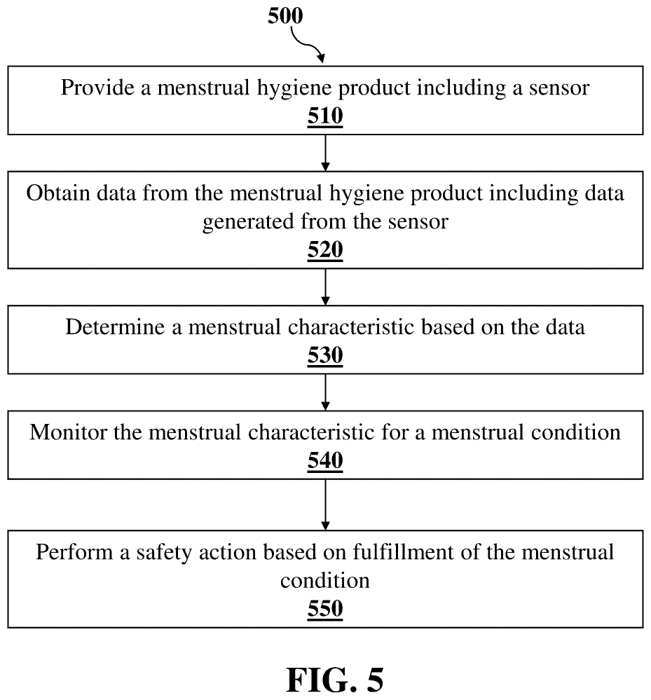

FIG. 5 is a flowchart of a method 500 of monitoring fluid menstrual fluid for diagnosis and treatment. The method 500 may be performed by any suitable system, apparatus, or device. For example, the method 500 may be implemented using the instrument 100 of FIG. 1, the environment 200 of FIG. 2, the menstruation monitoring application 300 of FIG. 3, or the computing system 700 of FIG. 7. Although illustrated with discrete blocks, the steps and operations associated with one or more blocks of the method 500 may be divided into additional blocks, combined into fewer blocks, or eliminated, depending on the particular implementation. For example, one or more of the operations described above with respect to the operational workflow 400 of FIG. 4 may be performed as part of the method 400.

The method 500 may include blocks 510, 520, 530, 540, and 550. At block 510, a menstrual hygiene product including a sensor may be provided. The menstrual hygiene product may be similar to and perform similar functions as the menstrual hygiene product 110 and 212 of FIGS. 1 and 2, respectively. The sensor may be similar to and perform similar functions as the sensor 112 and 214 of FIGS. 1 and 2, respectively. The menstrual hygiene product including the sensor allow the menstrual hygiene product to function in a similar manner as the instruments 100 and 210 of FIGS. 1 and 2, respectively.

At block 520, data may be obtained from the menstrual hygiene product including data generated from the sensor. In some embodiments, the data may include image data generated from an image of the menstrual hygiene product, weight data indicating a weight of the menstrual hygiene product, time data indicating a length of time that the menstrual hygiene product has been in use, resistance data indicating a resistance of the sensor, and/or biological data of the menstrual fluid.

At block 530, a menstrual characteristic may be determined based on the data. In some embodiments, the menstrual characteristic may include blood loss, saturation level of the menstrual hygiene product, time that the menstrual hygiene product has been in use, a presence of biomarkers in the menstrual fluid, or blood clot size. In some embodiments, the menstrual characteristic may be determined by an instrument such as the instruments 100 and 210 of FIGS. 1 and 2, respectively. In some embodiments, the menstrual characteristic may be determined by a user device such as the user device 220 of FIG. 2. In some embodiments the blood loss and/or the saturation level, may be determined from weight and/or resistance data.

At block 540, the menstrual characteristic may be monitored for a menstrual condition. In some embodiments, the menstrual condition may include a blood loss threshold, a saturation threshold, a time threshold, a presence of a specific biomarker in the menstrual fluid, a blood clot size threshold, and/or a medication release time. In some embodiments, the menstrual characteristic may be monitored by an instrument such as the instruments 100 and 210 of FIGS. 1 and 2, respectively. In some embodiments, the menstrual characteristic may be monitored by a user device such as the user device 220 of FIG. 2.

At block 550, a safety action may be performed based on fulfillment of the menstrual condition. In some embodiments, the safety action may include providing a notification on a user device. In some embodiments, the safety action may include releasing medicine at the menstrual hygiene product. In some embodiments, the safety action may be performed by an instrument such as the instruments 100 and 210 of FIGS. 1 and 2, respectively. In some embodiments, the safety action may be performed by a user device such as the user device 220 of FIG. 2.

Modifications, additions, or omissions may be made to the method 500 without departing from the scope of the disclosure. For example, the designations of different elements in the manner described is meant to help explain concepts described herein and is not limiting. Further, the method 500 may include any number of other elements or may be implemented within other systems or contexts than those described.

For example, the method 500 may further include establishing a baseline for the menstrual characteristic and the menstrual condition may include a deviation from the baseline for the menstrual characteristic. In some embodiments, the baseline may be established by an instrument such as the instruments 100 and 210 of FIGS. 1 and 2, respectively. In some embodiments, the baseline may be established by a user device such as the user device 220 of FIG. 2.

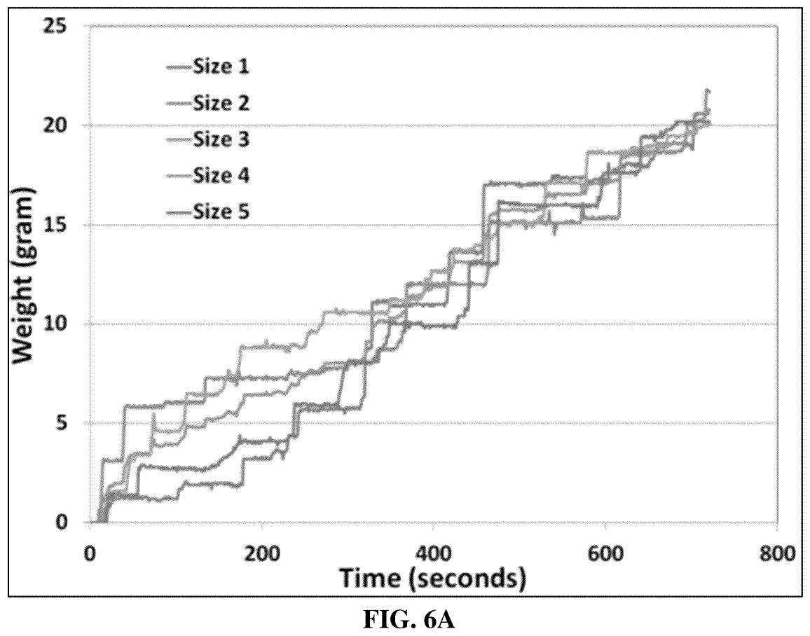

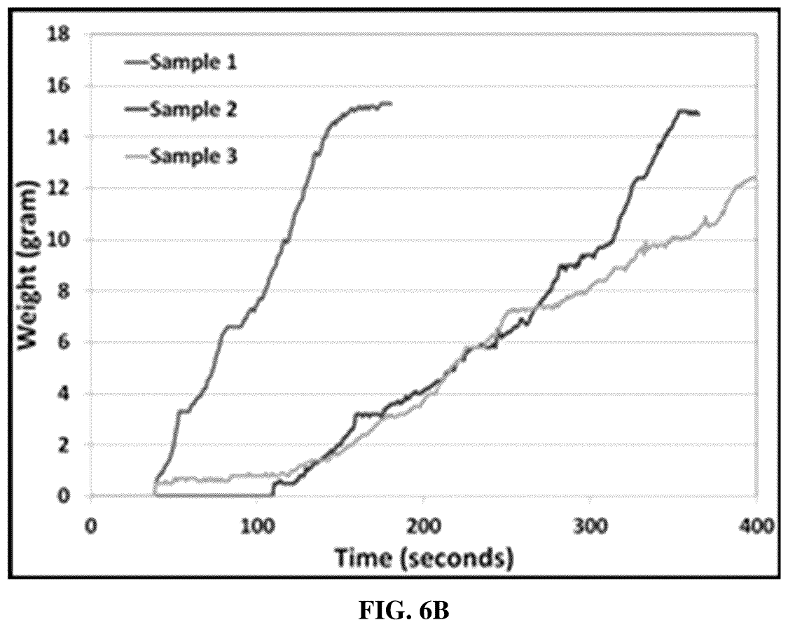

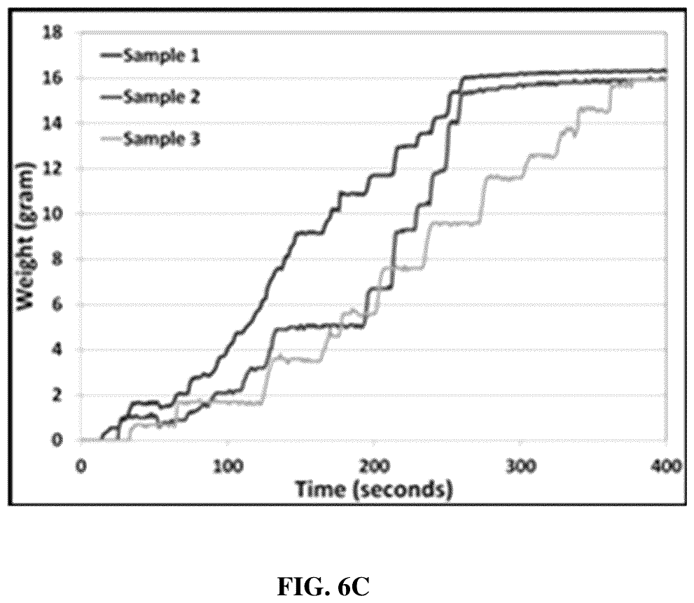

FIGS. 6A, 6B, and 6C respectively illustrate a first simulated experiment of measuring fluid absorption on a menstrual hygiene product utilizing a load cell incorporated in the menstrual hygiene product, a second simulated experiment of measuring fluid absorption on a menstrual hygiene product, and a third simulated experiment of measuring fluid absorption on varying sizes of menstrual hygiene products.

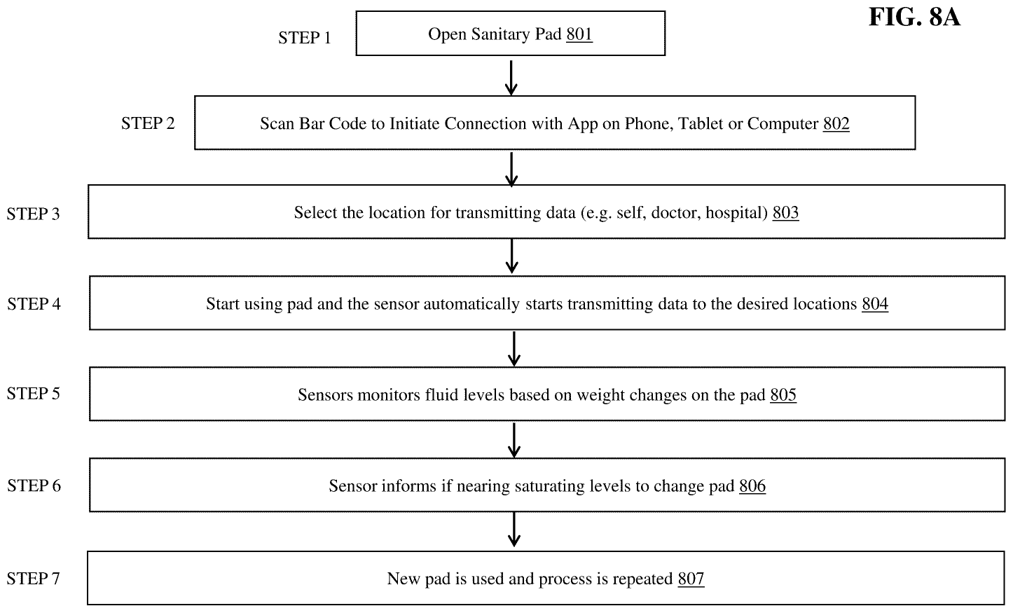

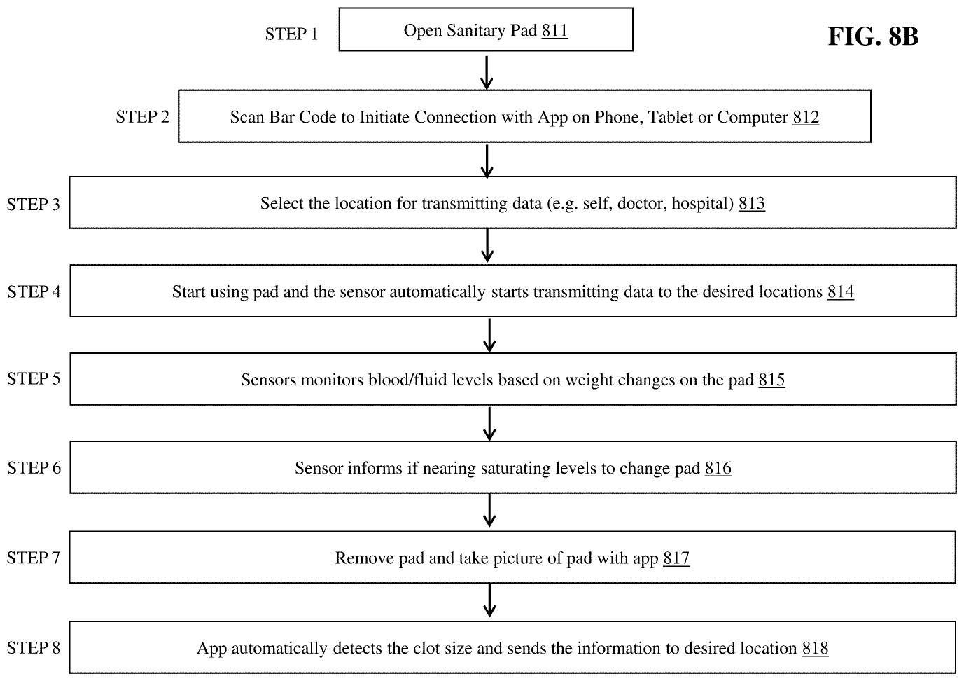

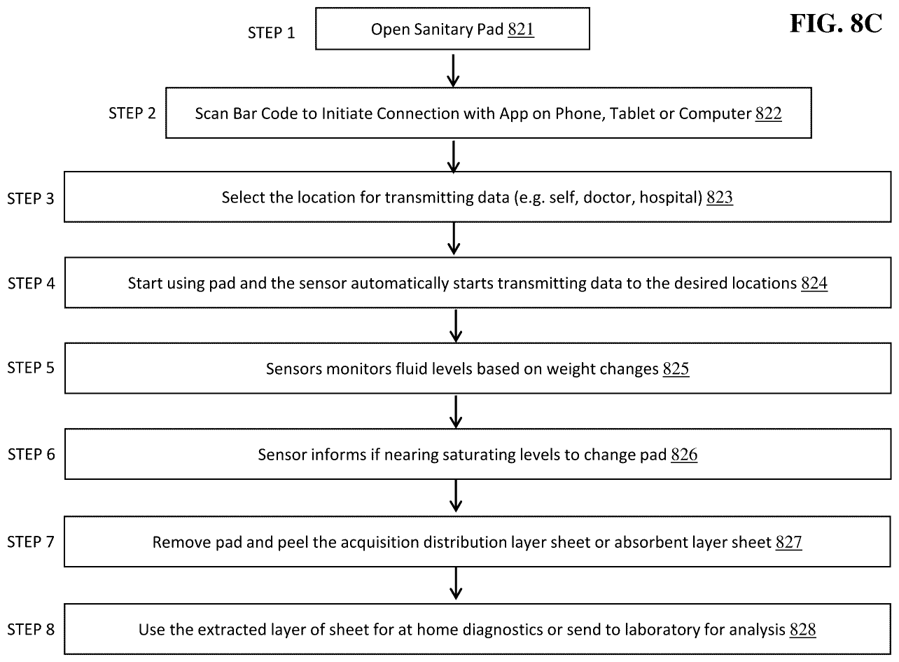

FIG. 8A shows an example of a process for the case of monitoring fluid absorption on the pad and how the sensor transmits information and the need to change the pad. First, the pad is opened (block 801) and the scan initiates the pairing of the pad with the app (block 802). Next, the patient selects to whom the data will be transmitted (block 803). Once the patient starts using the pad (block 804), the app automatically starts sending the status of the fluid levels on the pad based on the weight changes, electrical properties changes or other combinations that can be converted to fluid volume (block 805). Once the sensor detects the saturation levels it signals the patient to change the pad (block 806). As the next pad is used, the whole process is repeated and the patient can then accurately measure the volume of the fluids (block 807).

FIG. 8B shows an example of another process for monitoring blood loss and

clot size. First, the pad is opened (block 811) and the scan initiates the pairing of the pad with the app (block 812). Next, the patient selects to whom the data will be transmitted (block 813). Once the patient starts using the pad (block 814), the app automatically starts sending the status of the fluid levels on the pad based on the weight changes, electrical properties changes or other combinations that can be converted to fluid volume (block 815).

Once the sensor detects the saturation levels it signals the patient to change the pad (block 816). Imaging of the pad is performed to quantify the size of the blood clots (block 817). Once the image is taken, an app analysis and produces information on the clot sizes and the number of clots to automatically log that information (block 818).

FIG. 8C illustrates a process of the sensor integrated sanitary pad for diagnostic assays. Steps are performed as shown in blocks 821, 822, 823, 824, 825, and 826. The peelable/removable sheets (block 827) in the pad allow for downstream analysis of the samples for at home diagnostics or lab based including RNA, DNA, proteins, metabolites, pathogens, other biological entities among others (block 828).

In some embodiments, the pad/tampons can be further integrated with therapeutics to guide treatment and the sensors can allow tracking of blood loss to monitor the progress of the patient. Sensor can also be integrated with blood based diagnostics components (DNA, RNA, proteins, metabolites, pathogen, etc.). Customized pads made with tailor made treatments will accelerate the process of treatment. The sensors can be used for any kind of pads (synthetic, natural or hybrid) and for all sizes. The sensor types can also be mixed with different formulations depending on the requirements of the clinician for treatment.

In some embodiments, the present invention can provide information on blood loss using both off pad and on pad analysis. Real-time monitoring is on the pad while end point measurement and diagnosis is off the pad.

In some embodiments, the sensor can be made of both synthetic and natural materials that can biodegrade such as polylactic acid (PLA), poly(lactic-co-glycolic acid) (PLGA), poly(L-lactic acid) (PLLA), poly-γ-glutamic acid (PGA), polycaprolactone (PCL), etc. Similarly, plant-based polysaccharides (e.g., pectin, cellulose, alginate, dextran) and animal-derived materials (e.g., collagen, chitin, chitosan, dextran, shellac, gelatin, silk, chitosan) can be used. The biodegradables sensors have a biodegradable battery that is activated upon contact with fluid which enables the sensor to transfer the date from the pad via the app to the remote phone, tablet or computer of the patient, nurse or doctor. The sensors' data are observed by patients, nurses and doctors to decide on the next course of action for the treatment and monitor the progress.

In some embodiments, a method for preparing a personalized pad with medicines specific to treatment is provided. The pad can include: a sensor for on pad detection, a removable/peelable sheet for off pad detection; a camera coupled to an app to determine the blood clot sizes, wherein the app process the images and transmits the information from the sensor to the desired location.

In some embodiments, the pad can contain multiple sheets for detection different biological molecules (DNA, RNA, proteins, metabolites) and pathogens (bacteria, virus, fungi, etc.) beyond the blood cellular components; each layer of the pad can contain different sensors for different detection.

In some embodiments, the system can include a sensor for real-time monitoring of fluid or just biological molecules, pathogen or a combination of all. The sensor can be arranged as single, dual, multiple or array and at different places in the pad or tampon.

In some embodiments, the method can include: detecting changes in the patient and building a history of the patient and then comparing for diagnosing any differences between normal condition to determine the onset of menorrhagia.

In some embodiments, the method can include: detecting changes fluid absorbed onto the pad; and comparing the readings from the sensor to off pad analysis for biological molecules and pathogens and other conditions to build a correlation.

In some embodiments, the method can include: transmitting the data actively or passively to a device (phone, computer) capable of receiving the transmission.

In some embodiments, the method can include: detecting changes and transmitting the data actively or passively to a device (phone, computer) capable of receiving the transmission; using the data to decide the next course of treatment.

In some embodiments, the method can include: detecting changes; transmitting the data actively or passively to an app capable of receiving the transmission; using the data to decide the next course of treatment.

In some aspects, the method is to use the pad or tampon for treatment. In some aspects, the method can include: use of the pad and tampon with medicines, monitoring the blood loss; deciding the next mode of treatment; performing a second round of customized medicine on the pad or tampon for the desired treatment; repeating the entire process till blood loss is under control.

FIG. 7A illustrates a block diagram of an example computing system 700, according to at least one embodiment of the present disclosure. The computing system 700 may be configured to implement or direct one or more suitable operations described in the present disclosure. For example, the computing system 700 may be part of one or more of the elements of FIG. 1, FIG. 2, or FIG. 3 and/or be configured to perform one or more of the processes of FIG. 4, or the method of FIG. 5. The computing system 700 may include a processor 710, a memory 720, and a data storage 730. The processor 710, the memory 720, and the data storage 730 may be communicatively coupled.

In general, the processor 710 may include any suitable special-purpose or general-purpose computer, computing entity, or processing device including various computer hardware or software modules and may be configured to execute instructions stored on any applicable computer-readable storage media. For example, the processor 710 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 7, the processor 710 may include any number of processors configured to, individually or collectively, perform or direct performance of any number of operations described in the present disclosure. Additionally, one or more of the processors may be present on one or more different electronic devices, such as different servers.

In some embodiments, the processor 710 may be configured to interpret and/or execute program instructions and/or process data stored in the memory 720, the data storage 730, or the memory 720 and the data storage 730. In some embodiments, the processor 710 may fetch program instructions from the data storage 730 and load the program instructions in the memory 720. After the program instructions are loaded into memory 720, the processor 710 may execute the program instructions.

The memory 720 and the data storage 730 may include computer-readable storage media for carrying or having computer-executable instructions or data structures stored thereon. By way of example, and not limitation, such computer-readable storage media may include tangible or non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other non-transitory storage medium which may be used to carry or store particular program code in the form of computer-executable instructions or data structures and which may be accessed by a general-purpose or special-purpose computer. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007).

Combinations of the above may also be included within the scope of computer-readable storage media. Computer-executable instructions may include, for example, instructions and data configured to cause the processor 710 to perform a certain operation or group of operations.

Modifications, additions, or omissions may be made to the computing system 700 without departing from the scope of the present disclosure. For example, in some embodiments, the computing system 700 may include any number of other components that may not be explicitly illustrated or described.

FIG. 7B shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (uP), a microcontroller (C), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general-purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. For example, one or more neural network configurations may be used to perform the specific features and acts described above.

In some embodiments, a computer program product can include a non-transient, tangible memory device having computer-executable instructions that when executed by a processor, cause performance of methods described herein. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

The foregoing disclosure is not intended to limit the present disclosure to the precise forms or particular fields of use disclosed. As such, it is contemplated that various alternate embodiments and/or modifications to the present disclosure, whether explicitly described or implied herein, are possible in light of the disclosure. Having thus described embodiments of the present disclosure, it may be recognized that changes may be made in form and detail without departing from the scope of the present disclosure. Thus, the present disclosure is limited only by the claims.

In some embodiments, the different components, modules, engines, and services described herein may be implemented as objects or processes that execute on a computing system (e.g., as separate threads). While some of the systems and methods described herein are generally described as being implemented in software (stored on and/or executed by general purpose hardware), specific hardware implementations or a combination of software and specific hardware implementations are also possible and contemplated.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A method of monitoring menstrual fluid for diagnosis and treatment comprising:
providing a menstrual hygiene product including a sensor;
obtaining data from the menstrual hygiene product including data from the sensor;
determining a menstrual characteristic based on the data;
monitoring the menstrual characteristic for a menstrual condition; and
performing a safety action based on fulfillment of the menstrual condition.

2. The method of monitoring menstrual fluid for diagnosis and treatment of claim 1, wherein the data includes at least one of image data generated from an image of the menstrual hygiene product, weight data indicating a weight of the menstrual hygiene product, time data indicating a length of time that the menstrual hygiene product has been in use, resistance data indicating a resistance of the sensor, conductivity data indicating conductivity in the sensor, viscosity data indicating viscosity in the sensor or biological data of the menstrual fluid.

3. The method of monitoring menstrual fluid for diagnosis and treatment of claim 2, wherein the menstrual characteristic includes at least one of blood loss, saturation level of the menstrual hygiene product, time that the menstrual hygiene product has been in use, a presence of biomarkers in the menstrual fluid, presence of pathogens, or blood clot size.

4. The method of monitoring menstrual fluid for diagnosis and treatment of claim 3, wherein the menstrual condition includes at least one of: a blood loss threshold, a saturation threshold, a time threshold, a presence of a specific biomarker in the menstrual fluid, a blood clot size threshold, or a medication release time.

5. The method of monitoring menstrual fluid for diagnosis and treatment for treatment of claim 3, wherein at least one of the blood loss or the saturation level is determined based on at least one of the weight data or the resistance data.

6. The method of monitoring menstrual fluid for diagnosis and treatment of claim 1, wherein the safety action includes at least one of providing a notification on a user device or providing a notification at the menstrual hygiene product.

7. The method of monitoring menstrual fluid for diagnosis and treatment of claim 1, wherein the safety action includes releasing medicine at the menstrual hygiene product.

8. The method of monitoring menstrual fluid for diagnosis and treatment of claim 1, further comprising:
establishing a baseline for the menstrual characteristic; and
wherein the menstrual condition includes a deviation from the baseline for the menstrual characteristic.

9. A menstruation monitoring instrument, comprising:
a menstrual hygiene product; and
a sensor incorporated in the menstrual hygiene product, the sensor being configured to collect data from menstrual fluid contacting the menstrual hygiene product.

10. The menstruation monitoring instrument of claim 9, further comprising:
a camera incorporated in the menstrual hygiene product, or an external camera, the camera being configured to capture images of the menstrual fluid contacting the menstrual hygiene product.

11. The menstruation monitoring instrument of claim 9, further comprising:
a processor incorporated in the menstrual hygiene product, the processor being communicatively coupled to the sensor wherein the processor is configured to transmit the data collected by the sensor to a user device.

12. The menstruation monitoring instrument of claim 10, further comprising:
a processor incorporated in the menstrual hygiene product and communicatively coupled to the sensor and the camera, the processor being configured to transmit the data collected by the sensor to a user device and transmit image data from the camera to the user device.

13. The menstruation monitoring instrument of claim 9, wherein the sensor is a part of an array of sensors, wherein the array of sensors are incorporated into the menstrual hygiene product.

14. The menstruation monitoring instrument of claim 9, wherein the sensor is configured to collect at least one of weight data indicating a weight of the menstrual hygiene product, time data indicating a length of time that the menstrual hygiene product has been in use, resistance data indicating a resistance of the sensor, conductivity data indicating conductivity in the sensor, viscosity data indicating viscosity in the sensor or biological data of the menstrual fluid.

15. The menstruation monitoring instrument of claim 9, wherein the menstrual hygiene product is at least one of a tampon, a sanitary pad, period underwear, or a menstrual cup.

16. The menstruation monitoring instrument of claim 11, wherein the menstrual hygiene product is configured to release medicine in response to at least one of the following:
the sensor contacting menstrual fluid; or
a signal to dispense medicine being received at the processor; or
the processor receiving data from the sensor indicating that a menstrual condition has been fulfilled.

17. The menstruation monitoring instrument of claim 11, wherein the processor is configured to determine a menstrual characteristic based on the data, wherein the menstrual characteristic includes at least one of blood loss, saturation level of the menstrual hygiene product, time that the menstrual hygiene product has been in use, a presence of biomarkers in the menstrual fluid, or blood clot size.

18. The menstruation monitoring instrument of claim 17, wherein the processor is configured to monitor a menstrual characteristic for a menstrual condition, wherein the menstrual condition includes at least one of a blood loss threshold, a saturation threshold, a time threshold, a presence of a specific biomarker in the menstrual fluid, a blood clot size threshold, or a medication release time.

19. A device comprising:
one or more non-transitory computer-readable media configured to store instructions; and
a processor coupled to the computer-readable media and configured to execute the instructions to perform operations, the operations comprising:
obtaining data including data collected from a sensor;
determining a menstrual characteristic based on the data;
monitoring the menstrual characteristic for a menstrual condition; and
performing a safety action based on an occurrence of the menstrual condition.

20. The device of claim 19, wherein the operations further comprise:
establishing a baseline for the menstrual characteristic; and
wherein the menstrual condition includes a deviation from the baseline for the menstrual characteristic.
